(19) United States
(12) Patent Application Publication
(10) Pub. No.: US 2015/0351691 A1
(43) Pub. Date: Dec. 10, 2015

(54) NANOSCALE WIRE PROBES

(71) Applicant: President and Fellows of Harvard College, Cambridge, MA (US)

(72) Inventors: Charles M. Lieber, Lexington, MA (US); Or A. Shemesh, Somerville, MA (US); Ruixuan Gao, Cambridge, MA (US)

(21) Appl. No.: 14/423,340

(22) PCT Filed: Aug. 21, 2013
(86) PCT No.: PCT/US2013/055910

§ 371 Date: Feb. 23, 2015

Related U.S. Application Data
(60) Provisional application No. 61/693,183, filed on Aug. 24, 2012.

(51) Int. Cl. A61B 5/00; A61N 1/05; A61B 5/145; A61B 5/04; A61B 5/1473

(52) U.S. Cl. CPC A61B 5/6847; A61B 5/04; A61B 5/1473; A61B 5/14539; A61N 1/05; A61B 2562/0285

(57) ABSTRACT

The present invention generally relates to nanoscale wires and, in particular, to probes comprising nanoscale wires for use in determining electrical and/or chemical properties in a tissue or other material. For example, in certain embodiments, a probe comprising nanoscale wires may be inserted into an electrically-active tissue, such as the heart or the brain, and the nanoscale wires may be used to determine electrical properties of the tissue, e.g., action potentials or other electrical activity. In addition, in some embodiments, a nanoscale wire may be modified to determine chemical properties of a tissue. A probe comprising such nanoscale wires can be inserted into a tissue (not necessarily electrically active) to determine various properties, e.g., chemical or mechanical properties. In addition, in some embodiments, a probe is provided that can be used to stimulate tissues, e.g., by providing electrical stimuli via one or more nanoscale wires. Still other embodiments are generally directed to systems and methods of making, using, or promoting such probes, kits involving such probes, and the like.

### RELATED APPLICATIONS

This application claims the benefit of U.S. Provisional Patent Application Ser. No. 61/693,183, filed Aug. 24, 2012, entitled “Nanoscale Wire Probes,” incorporated herein by reference in its entirety.

### GOVERNMENT FUNDING

Research leading to various aspects of the present invention was sponsored, at least in part, by the National Institutes of Health (NIH), Grant No. 5DP1OD003900. The U.S. Government has certain rights in the invention.

### FIELD

The present invention generally relates to nanoscale wires and, in particular, to probes comprising nanoscale wires for use in determining properties such as electrical and/or chemical properties in a tissue or other biological system.

### BACKGROUND

Interest in nanotechnology, in particular sub-microelectronic technologies such as semiconductor quantum dots and nanowires, has been motivated by the challenges of chemistry and physics at the nanoscale, and by the prospect of utilizing these structures in electronic and related devices. Nanoscopic articles might be well-suited for transport of charge carriers and excitons (e.g. electrons, electron pairs, etc.) and thus may be useful as building blocks in nanoscale electronics applications.

Nanoscale wires having selectively functionalized surfaces have been described in, e.g., U.S. Pat. No. 7,129,554, issued Oct. 31, 2006, entitled “Nanosensors,” by Lieber, et al., incorporated herein by reference in its entirety. Functionalization of a nanoscale wire may permit interaction of the functionalized nanoscale wire with various entities, such as molecular entities, and the interaction may induce a change in a property of the functionalized nanoscale wire, which provides a mechanism for a nanoscale sensor device for detecting the presence or absence of an analyte suspected to be present in a sample. However, larger structures, such as tissues, have not been studied using such nanoscale wires, in part due to the difficulty of accurately positioning nanoscale wires within such tissues.

### SUMMARY

The present invention generally relates to nanoscale wires and, in particular, to probes comprising nanoscale wires for use in determining properties such as electrical and/or chemical properties in a tissue or other biological system. The subject matter of the present invention involves, in some cases, interrelated products, alternative solutions to a particular problem, and/or a plurality of different uses of one or more systems and/or articles.

In one aspect, the present invention is directed to an article for insertion into a tissue or other material. In one set of embodiments, the article includes a substrate constructed and arranged for insertion into tissue, a plurality of nanoscale wires, and a plurality of electrical connectors in electrical communication with the plurality of nanoscale wires.

The present invention, in another aspect, is directed to a method. According to one set of embodiments, the method includes an act of inserting a substrate comprising a plurality of nanoscale wires into a tissue of a subject, or into another suitable material. In another set of embodiments, the method includes an act of externally delivering an electrical stimulus to a tissue within a subject, or another suitable material, via a nanoscale wire inserted therein. The method, in yet another set of embodiments, includes an act of determining a property of a nanoscale wire inserted into a tissue within a subject, or another suitable material.

In one set of embodiments, the present invention is generally directed to using a nanoscale wire to measure the heart rate of a subject. The subject may be human in some cases.

In another aspect, the present invention encompasses methods of making one or more of the embodiments described herein, for example, a probe comprising one or more nanoscale wires. In still another aspect, the present invention encompasses methods of using one or more of the embodiments described herein, for example, a probe comprising one or more nanoscale wires.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a probe in accordance with one embodiment of the invention;

FIGS. 2A-2E illustrate various substrates for nanoscale wires, in accordance with certain embodiments of the invention;

FIGS. 3A-3B illustrate a probe in accordance with one embodiment of the invention;

FIGS. 4A-4B illustrate various nanowires on a probe, in another embodiment of the invention;

FIGS. 5A-5B illustrate a nanowire angled from the surface of a probe, in yet another embodiment of the invention; and

FIGS. 6A-6C illustrate a probe and data obtained from the probe, in accordance with still another embodiment of the invention.

### DETAILED DESCRIPTION

The present invention generally relates to nanoscale wires and, in particular, to probes comprising nanoscale wires for use in determining electrical and/or chemical properties in a tissue or other material. For example, in certain embodiments, a probe comprising nanoscale wires may be inserted into an electrically-active tissue, such as the heart or the brain, and the nanoscale wires may be used to determine electrical properties of the tissue, e.g., action potentials or other electrical activity. In addition, in some embodiments, a nanoscale wire may be modified to determine chemical properties of a tissue. A probe comprising such nanoscale wires can be inserted into a tissue (not necessarily electrically active) to determine various properties, e.g., chemical or mechanical properties. In addition, in some embodiments, a probe is provided that can be used to stimulate tissues, e.g., by providing electrical stimuli via one or more nanoscale wires. Still other embodiments are generally directed to systems and methods of making, using, or promoting such probes, kits involving such probes, and the like.

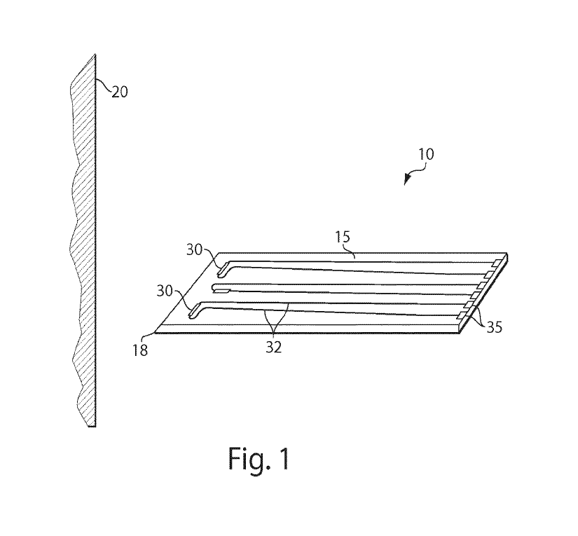

Turning first to FIG. 1, an example of an embodiment of the invention useful for determine a property of a biological tissue, and/or electrically stimulating the biological tissue, is now described. However, this is by way of example only, and as discussed in detail below, in other embodiments, other configurations may also be used.

In FIG. 1, probe 10 includes a substrate 15 having a tip 18 constructed and arranged for insertion into tissue 20. In this figure, tip 18 includes an angled portion that allows substrate 15 to be inserted more easily into tissue 20, although the tip may also be of a wide variety of shapes and/or sizes, as discussed in detail below. In addition, substrate 15 in this figure is substantially planar, although the substrate need not be in other embodiments. In addition, substrate 15 may be formed out of any suitable materials, including silicon, polymers, glass, biodegradable materials such as silk, or the like.

Probe 10 may also include one or more nanoscale wires 30, which may be in electrical communication via leads 32 with one or more electrical connectors 35 at an end of probe 10. One or more than one nanoscale wire may be in electrical communication with a given electrical connector. Although only a small number of nanoscale wires 30 are illustrated in FIG. 1, this is by way of example only, and in other embodiments, other numbers of nanoscale wires may be present on one or more exposed surfaces of probe 10. Nanoscale wires 30 may also be positioned in any suitable distribution on the surface of probe 10, e.g., in an ordered array, randomly positioned, etc.

The nanoscale wires on probe 10 may be, for example, semiconductor nanowires, carbon nanotubes, or other nanoscale wires such as those discussed below. In some cases, one or more of the nanoscale wires may be a kinked nanowire. If more than one nanoscale wire is present, the nanoscale wires may independently be the same or different. For example, the nanoscale wires may have the same, or different, shapes, lengths, sizes, diameters, materials, electrical configurations, etc.

Tissue 20 may be any suitable tissue which is to be determined and/or stimulated, for example, brain tissue, heart tissue, etc. In some cases, tissue 20 is electrically-active, although in other embodiments, tissue 20 is not necessarily electrically active. At least a portion of probe 10 can be inserted into tissue 20, for example, to deliver an electrical stimulus to at least a portion of tissue 20, and/or to determine a property, such as an electrical, chemical, and/or mechanical property, of at least a portion of tissue 20. In addition, in some cases, more than one such probe may be inserted into tissue 20, e.g., sequentially or simultaneously.

After insertion, one or more of the nanoscale wires may be determined and/or stimulated. For example, using electrical connectors 35, an electrical property of a nanoscale wire may be determined, and used to determine a property of cells or tissues surrounding the nanoscale wire. The property to be determined may be, for example, an electrical property, a chemical property, a mechanical property, etc. The determined property may be analyzed or recorded for later use. As a non-limiting example, the nanoscale wire may form a gate of a field-effect transistor, and an electrical property such as conductance, resistance, impedance, etc. of the nanoscale wire may thus be determined to determine a suitable property of the surrounding cells or tissue.

As another example, an electrical signal, such as voltage or current, may be applied to one or more electrical connectors 35, e.g., to cause a nanoscale wire to experience an electrical signal, which may be transmitted from the nanoscale wire to cells or tissues surrounding the nanoscale wire. For example, one or more than one electrical signals (e.g., action potentials, or other stimulatory or inhibitory signals, etc.) may be applied via the electrical connectors to a suitable portion of the tissue to cause an effect, e.g., in a living organism. In addition, in some embodiments, the same probe may be used for both determination and stimulation of a tissue.

In addition, in some embodiments, the probe may be inserted into other biological systems, or even other materials. For example, in some embodiments, a probe as discussed herein may be inserted into an organism, an artificial tissue, an inorganic material, a polymeric material, or the like, e.g., to determine a suitable property, or in some cases, to provide an electrical stimulus. Accordingly, in the discussions herein, it should be understood that the discussions of insertion into tissue is by way of example only, and in other embodiments, the probe may be inserted into other biological systems, or other materials.

The above discussion is a non-limiting example of one embodiment of the present invention, describing a probe that can be at least partially inserted into tissue, e.g., for stimulating the tissue and/or for determining a property of the tissue. However, other embodiments are also possible, e.g., for insertion into tissues, biological systems, other materials, etc. Thus, more generally, various aspects of the invention are directed to various systems comprising nanoscale wires for use in determining properties of a tissue or other system, and methods of use thereof. Any suitable property may be determined and/or recorded, e.g., electrical properties, chemical properties, mechanical properties, etc.

One aspect of the present invention is generally directed to a probe for insertion into a tissue, or other material. The probe can be fully or partially inserted into the tissue or other material. The probe may be used to determine a property of the tissue or other material, and/or provide an electrical signal to the tissue, or other material. This may be achieved using one or more nanoscale wires on the probe.

The probe itself may be formed from any suitable substrate. In some cases, the substrate is biocompatible and/or biodegradable, although the substrate need not be. For example, the substrate may comprise a substrate comprising a semiconductor (e.g., Si, Ge, GaAs, etc.), a metal, a glass, a polymer (e.g., polyethylene, polypropylene, poly(ethylene terephthalate), polydimethylsiloxane, or the like). Additional examples of such substrates, and techniques for placing nanoscale wires on such substrates, include, but are not limited to, nanoimprint lithography, fluid-directed assembly, Langmuir-Blodgett (LB) trough techniques, sliding substrate techniques, and the like. See, for example, U.S. patent application Ser. No. 10/995,075, filed Nov. 22, 2004, entitled “Nanoscale Arrays, Robust Nanostructures, and Related Devices,” by Whang, et al., published as 2005/0253137 on Nov. 17, 2005; or International Patent Application No. PCT/US2007/008540, filed Apr. 6, 2007, entitled “Nanoscale Wire Methods and Devices,” by Lieber et al., published as WO 2007/145701 on Dec. 21, 2007, each incorporated herein by reference in its entirety.

In one set of embodiments, the substrate is biocompatible. Typically, a biocompatible substrate can be partially or completely inserted into a tissue for an extended period of time (e.g., days or longer). A biocompatible substrate may be formed out of a material that does not induce an adverse immunological or biological reaction that reduces or eliminates functioning of the probe, for example, by chemically attacking the probe, forming a fibrous capsule around the probe, degrading tissue around the probe, etc.

The substrate may also be biodegradable in some cases. A biodegradable substrate may be formed from a substance that, when implanted into a tissue, begins to degrade or dissolve, for example, due to temperature, moisture, enzymes, etc. that may be present within the tissue. For example, the substrate may comprise or consist essentially of polymers such as poly(lactic acid) and/or poly(glycolic acid) that degrade or dissolve via hydrolysis. As another example, a biodegradable substrate may comprise or consist essentially of silk, which can degrade or dissolve upon exposure to various proteolytic enzymes such as chymotrypsin, actinase, or carboxylase.

The substrate may be planar or substantially define a plane, or the substrate may be non-planar or curved (i.e., a surface that can be characterized as having a finite radius of curvature). The substrate may also be a flexible substrate in some cases, e.g., the substrate may be able to bend or flex. For example, a flexible substrate may be bent or distorted by a volumetric displacement of at least about 5%, about 10%, or about 20% (relative to the undisturbed volume), without causing cracks and/or breakage of the substrate, i.e., the substrate can be distorted such that about 5%, about 10%, or about 20% of the mass of the substrate has been moved outside the original surface perimeter of the substrate, without causing failure of the flexible substrate (e.g., by breaking or cracking of the substrate). In some cases, the substrate may be bent or flexed as described above by an ordinary human being without the use of tools, machines, mechanical device, excessive force, or the like. Non-limiting examples of flexible substrates include polymers, fibers, or the like, e.g., as discussed herein.

In one set of embodiments, the substrate, or a portion thereof (e.g., the tip of the substrate) may be constructed and arranged for insertion into tissue (or another material). For example, the substrate may be shaped such that the substrate, or a portion thereof, may be pushed into a tissue without excessive force. In some embodiments, the substrate may be shaped such that an ordinary person can at least partially insert the probe into the tissue without any tools, machines, mechanical device, excessive force, etc., and without substantially deforming or damaging the tissue. Thus, for instance, the substrate may have an end that comes to a single point or a sharp knife or blade edge, or the substrate may have other shapes that allow it to be readily inserted into tissue.

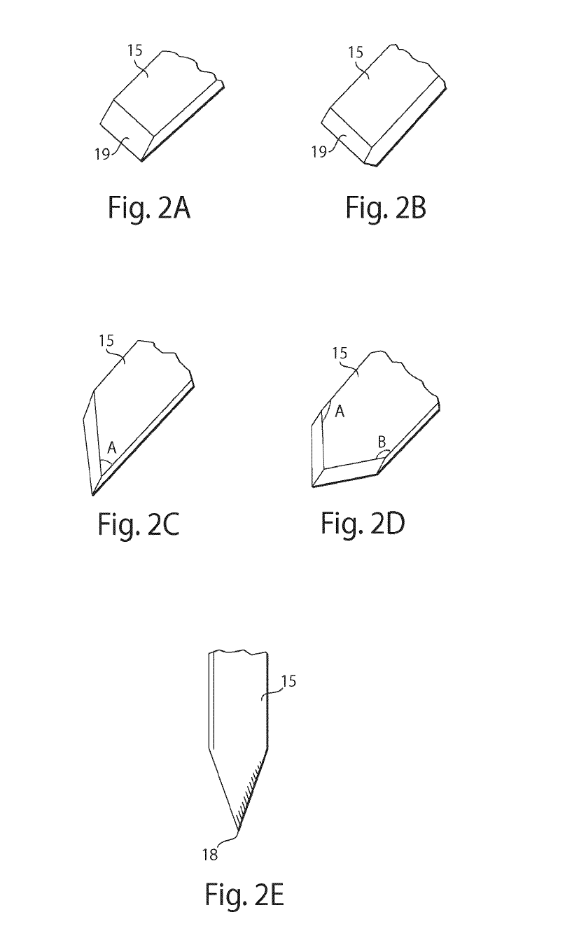

Non-limiting examples of such substrates may be seen in FIG. 2. In one set of embodiments, substrate 15 may be substantially planar, and include a sharpened edge region 19, which may be beveled (FIG. 2A) or double-beveled (FIG. 2B). In addition, in some cases, substrate 15 itself may also be angled for insertion into a tissue, e.g., to facilitate entry. Non-limiting examples are shown in FIGS. 2C and 2D, where angles A and B can be any suitable angle (e.g., a non-right angle), and can be the same or different in FIG. 2D. For instance, in FIG. 2C, the substrate is constructed and arranged to form an edge defined by acute angle A. Any suitable acute angle may be used. For example, the angle may be less than about 90°, less than about 80°, less than about 70°, less than about 60°, less than about 50°, less than about 45°, less than about 40°, less than about 30°, etc. In FIG. 2D, there are two such edges, defined by obtuse angles A and B, which may be symmetrically or nonsymetrically arranged on substrate 15. In addition, other shapes for region 19 may also be used in other embodiments, for example, one or more curved edges. Combinations of these and/or other shapes are also possible in yet other embodiments.

In some embodiments, the substrate has a thickness or shortest cross-sectional dimension of no more than about 1 mm, no more than about 500 micrometers, no more than about 300 micrometers, no more than about 100 micrometers, no more than about 50 micrometers, no more than about 30 micrometers, or no more than about 10 micrometers. The substrate may also have a width, in certain embodiments of the invention, of no more than about 1 mm, no more than about 500 micrometers, no more than about 300 micrometers, no more than about 200 micrometers, no more than about 100 micrometers, no more than about 50 micrometers, no more than about 30 micrometers, no more than about 20 micrometers, or no more than about 10 micrometers. In some cases, one or more of the dimensions of the substrate (length, width, and height) may each be independently chosen to be no more than about 1 mm, no more than about 500 micrometers, no more than about 300 micrometers, no more than about 100 micrometers, no more than about 50 micrometers, no more than about 30 micrometers, or no more than about 10 micrometers. The substrate may also have a width, in certain embodiments of the invention, of no more than about 1 mm, no more than about 500 micrometers, no more than about 300 micrometers, no more than about 200 micrometers, no more than about 100 micrometers, no more than about 50 micrometers, no more than about 30 micrometers, no more than about 20 micrometers, or no more than about 10 micrometers.

In one set of embodiments, the length (x dimension) of the probe may be chosen to be approximately as long as the recording depth, e.g., within a factor of +/−25%, +/−15%, +/−10%, or +/−5%. The width (y dimension, respectively) may, in some cases, be chosen to be less than about 1 mm, or other dimensions as discussed herein. As a specific non-limiting example, to determine a rat cortex, having a thickness of roughly 1000 micrometers, the length of the probe may also be chosen to be about 1000 micrometers, and the width and thickness may be chosen to roughly imitate a glass micropippete or a patch pipette, e.g., of less than about 200 micrometers.

In addition, the substrate may be non-planar in some cases, e.g., curved as previously discussed. For example, the substrate may be substantially U-shaped, or the substrate may be constructed and arranged to come to an end in one or more sharp points. For instance, in FIG. 2E, substrate 15 tapers to a single point 18, for ease of insertion into the tissue. Thus, substrate 15 may be cylindrical, or substrate 15 may have a shape and/or size similar to a hypodermic needle. In some embodiments, substrate 15 may have an outer diameter of no more than about 5 mm, no more than about 4 mm, no more than about 3 mm, no more than about 2 mm, no more than about 1 mm, no more than about 0.9 mm, no more than about 0.8 mm, no more than about 0.7 mm, no more than about 0.6 mm, no more than about 0.5 mm, no more than about 0.4 mm, no more than about 0.3 mm, or no more than about 0.2 mm.

Positioned on one or more surfaces of the substrate may be one or more nanoscale wires, which may be the same or different from each other. Non-limiting examples of such nanoscale wires are discussed in detail below, and include, for instance, semiconductor nanowires, carbon nanotubes, or the like. The nanoscale wires may also be straight, or kinked in some cases. In some embodiments, one or more of the nanoscale wires may form at least a portion of a transistor, such as a field-effect transistor, e.g., as is discussed in more detail below. The nanoscale wires may be distributed on only one surface, or more than one surface in some cases (for example, the front and back of a substantially planar substrate). The nanoscale wires may be distributed on the surface in any suitable configuration, for example, in an ordered array or randomly distributed. In some cases, the nanoscale wires are distributed such that an increasing concentration of nanoscale wires can be found towards the portion of the substrate that is inserted into the tissue.

In some cases, some or all of the nanoscale wires are individually electronically addressable. For instance, in some cases, at least about 10%, at least about 20%, at least about 30%, at least about 40%, at least about 50%, at least about 60%, at least about 70%, at least about 80%, at least about 90%, or substantially all of the nano scale wires may be individually electronically addressable. In some embodiments, an electrical property of a nanoscale wire can be individually determinable (e.g., being partially or fully resolvable without also including the electrical properties of other nanoscale wires), and/or such that the electrical property of a nanoscale wire may be individually controlled (for example, by applying a desired voltage or current to the nanoscale wire, for instance, without simultaneously applying the voltage or current to other nanoscale wires). In other embodiments, however, at least some of the nanoscale wires can be controlled within the same electronic circuit (e.g., by incorporating the nanoscale wires in series and/or in parallel), such that the nanoscale wires can still be electronically controlled and/or determined.

In various embodiments, more than one nanoscale wire may be present on the substrate. The nanoscale wires may each independently be the same or different. For example, the substrate can comprise at least 5 nanoscale wires, at least about 10 nanoscale wires, at least about 15 nanoscale wires, at least about 20 nanoscale wires, at least about 25 nanoscale wires, at least about 30 nanoscale wires, at least about 50 nanoscale wires, at least about 100 nanoscale wires, at least about 300 nanoscale wires, at least about 1000 nanoscale wires, etc.

In addition, in some embodiments, there may be a relatively high density of nanoscale wires on the substrate, or at least a portion of the substrate. The nanoscale wires may be distributed uniformly or non-uniformly on the substrate. In some cases, the nanoscale wires may be distributed at an average density of at least about 5 wires/mm², at least about 10 wires/mm², at least about 30 wires/mm², at least about 50 wires/mm², at least about 75 wires/mm², at least about 100 wires/mm², at least about 300 wires/mm², at least about 500 wires/mm², at least about 750 wires/mm², at least about 1000 wires/mm², etc. In certain embodiments, the nanoscale wires are distributed on the substrate such that the average separation between a nanoscale wire and its nearest neighboring nanoscale wire is less than about 2 mm, less than about 1 mm, less than about 500 micrometers, less than about 300 micrometers, less than about 100 micrometers, less than about 50 micrometers, less than about 30 micrometers, or less than about 10 micrometers.

Some or all of the nanoscale wires may be in electrical communication with one or more electrical connectors via one or more conductive pathways. The electrical connectors may be positioned on any portion of the substrate, e.g., in an edge, region, or end of the substrate that is not inserted into the tissue. The electrical connectors may be made out of any suitable material that allows transmission of an electrical signal. For example, the electrical connectors may comprise gold, silver, copper, aluminum, tantalum, titanium, nickel, tungsten, chromium, palladium, etc. In some cases, the electrical connectors have an average cross-section of less than about 10 micrometers, less than about 8 micrometers, less than about 6 micrometers, less than about 5 micrometers, less than about 4 micrometers, less than about 3 micrometers, less than about 2 micrometers, less than about 1 micrometer, etc.

In some embodiments, the electrical connectors and conductive pathways can be used to determine a property of a nanoscale wire (for example, an electrical property or a chemical property as is discussed herein), and/or to direct an electrical signal to a nanoscale wire, e.g., to electrically stimulate cells proximate the nanoscale wire. The conductive pathways can form an electrical circuit that is internally contained within the substrate, and/or that extends externally of the substrate, e.g., such that the electrical circuit is in electrical communication with an external electrical system, such as a computer or a transmitter (for instance, a radio transmitter, a wireless transmitter, an Internet connection, etc.). Any suitable pathway conductive pathway may be used, for example, pathways comprising metals, semiconductors, conductive polymers, or the like.

Furthermore, more than one conductive pathway may be used in certain embodiments. For example, multiple conductive pathways can be used such that some or all of the nanoscale wires on the substrate may be electronically individually addressable, as previously discussed. However, in other embodiments, more than one nanoscale wire may be addressable by a particular conductive pathway. In addition, in some cases, other electronic components may also be present on the substrate, e.g., as part of a conductive pathway or otherwise forming part of an electrical circuit. Examples include, but are not limited to, transistors such as field-effect transistors or bipolar junction transistors, resistors, capacitors, inductors, diodes, integrated circuits, etc. In certain cases, some of these may also comprise nanoscale wires. For example, in some embodiments, two sets of electrical connectors and conductive pathways, and a nanoscale wire, may be used to define a transistor such as a field effect transistor, e.g., where the nanoscale wire defines the gate. As mentioned, the environment in and/or around the nanoscale wire can affect the ability of the nanoscale wire to function as a gate.

As mentioned, in various embodiments, one or more electrodes, electrical connectors, and/or conductive pathways may be positioned in electrical and/or physical communication with the nanoscale wires. These can be patterned to be in direct physical contact the nanoscale wire and/or there may be other materials that allow electrical communication to occur. Metals may be used due to their high conductance, e.g., such that changes within electrical properties obtained from the conductive pathway may be related to changes in properties of the nanoscale wire, rather than changes in properties of the conductive pathway. However, in other embodiments, other types of electrode materials are used, in addition or instead of metals.

A wide variety of metals may be used in various embodiments of the invention, for example in an electrode, electrical connector, conductive pathway, etc. As non-limiting examples, the metals may include one or more of aluminum, gold, silver, copper, molybdenum, tantalum, titanium, nickel, tungsten, chromium, palladium, as well as any combinations of these and/or other metals. In some cases, the metal may be chosen to be one that is readily introduced, e.g., using techniques compatible with lithographic techniques. For example, in one set of embodiments, lithographic techniques such as e-beam lithography, photolithography, X-ray lithography, extreme ultraviolet lithography, ion projection lithography, etc. can be used to pattern or deposit one or more metals on a substrate.

Additional processing steps can also be used to define or register the electrode, electrical connector, or conductive pathway in some cases. Thus, for example, the thickness may be less than about 5 micrometers, less than about 4 micrometers, less than about 3 micrometers, less than about 2 micrometers, less than about 1 micrometer, less than about 700 nm, less than about 600 nm, less than about 500 nm, less than about 300 nm, less than about 200 nm, less than about 100 nm, less than about 80 nm, less than about 50 nm, less than about 30 nm, less than about 10 nm, less than about 5 nm, less than about 2 nm, etc. The thickness of the electrode may also be at least about 10 nm, at least about 20 nm, at least about 40 nm, at least about 60 nm, at least about 80 nm, or at least about 100 nm. For example, the thickness of an electrode may be between about 40 nm and about 100 nm, between about 50 nm and about 80 nm.

In some embodiments, more than one metal may be used. The metals can be deposited in different regions or alloyed together, or in some cases, the metals may be layered on top of each other, e.g., layered on top of each other using various lithographic techniques. For example, a second metal may be deposited on a first metal, and in some cases, a third metal may be deposited on the second metal, etc. Additional layers of metal (e.g., fourth, fifth, sixth, etc.) can also be used in some embodiments. The metals may all be different, or in some cases, some of the metals (e.g., the first and third metals) may be the same. Each layer may independently be of any suitable thickness or dimension, e.g., of the dimensions described above, and the thicknesses of the various layers may independently be the same or different.

In one aspect, a nanoscale wire (e.g., a nanotube or a nanowire) may be held at an angle away from the surface of the substrate, for example, by a suitable holding member. See, e.g., U.S. Provisional Patent Application Ser. No. 61/642,111, filed May 3, 2012, entitled “Nanoscale Sensors for Intracellular and Other Applications,” by Lieber, et al., incorporated herein by reference in its entirety. In some embodiments, the holding member comprises a polymer, such as a photoresist. For example, the photoresist can be chosen for its ability to react to light to become substantially insoluble (or substantially soluble, in some cases) to a photoresist developer. For instance, photoresists that may be used within a polymeric construct include, but are not limited to, SU-8, S1805, LOR 3A, poly(methyl methacrylate), poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, or the like. These and many other photoresists are available commercially. In some embodiments, one or more portions of the photoresist can be exposed to light (visible, UV, etc.), electrons, ions, X-rays, etc. (e.g., projected onto the photoresist), and the exposed portions may be etched away (e.g., using suitable etchants, plasma, etc.) to produce the pattern.

In some embodiments, only a portion of the nanoscale wire is held by the holding member, e.g., such that only one end of the nanoscale wire is supported by the holding member. For example, the nanoscale wire may comprise a free portion (not in physical contact with the holding member) and a held portion (in physical contact with the holding member), such that the free portion is at least about 10%, at least about 20%, at least about 30%, at least about 40%, at least about 50%, at least about 60%, at least about 70%, at least about 80%, or at least about 90% of the nanoscale wire. In addition, more than one free portion and/or held portion may be present in some embodiments.

The holding member may hold the nanoscale wire at any suitable angle away from the substrate. For example, the angle can be about 10°, about 20°, about 30°, about 40°, about 50°, about 60°, about 70°, about 80°, or about 90° (i.e., vertically positioned relative to the substrate). If more than one nanoscale wire is held by the holding member, the nanoscale wires can be held at the same or different angles.

The holding member may be angled away from the substrate, in one set of embodiments, by depositing two or more dissimilar metals on the holding member that may warp or bend, thereby causing the holding member to warp away from the substrate. Examples of such metals are disclosed herein. In some (but not all) embodiments, the metals can also be used for one or more electrodes, e.g., as discussed herein. As a specific non-limiting example, chromium and palladium may be layered or deposited on each other in such a way that stresses occur between the metals, thereby causing warping or bending. As another non-limiting example, copper and chromium may be layered or deposited on each other to cause warping or bending. The amount and type of stress can also be controlled, e.g., by controlling the thicknesses of the layers. For example, relatively thinner layers may be used to increase the amount of warping that occurs.

In some cases, lengths of metals may also be used to control the amount of bending or warping, in addition to and/or instead of controlling the thicknesses of the metals. For example, by using longer lengths in the holding member, larger angles and/or heights of the nanoscale wire, relative to the substrate, may be achieved. For example, the effect of a relatively small deflection in two dissimilar metals may be geometrically increased due to longer lengths of metals that are bent or warped, even if at any one location, the amount of deflection or stress is relatively small.

Without wishing to be bound by any theory, it is believed that layering metals having a difference in stress (e.g., film stress) with respect to each other may, in some cases, cause stresses within the metal, which can cause bending or warping as the metals seek to relieve the stresses. For example, a first layer having a first film stress deposited on a second layer having a second film stress greater than the first film stress may cause bending or warping towards the direction of the second layer. In certain embodiments, the deposition of stressed metals may occur at one or more specific locations, e.g., to cause specific warpings to occur, e.g., at certain places, which may be used to cause the holding member to be deformed into a particular shape or configuration. For example, a “line” of such mismatches can be used to cause an intentional bending or folding along the line of the holding member.

The holding member is positioned on a substrate in certain embodiments. The substrate may be chosen to be one that can be used for lithographic techniques such as e-beam lithography or photolithography, or other lithographic techniques including those discussed herein. For example, the substrate can comprise or consist essentially of a semiconductor material such as silicon, although other substrate materials (e.g., a metal) can also be used. Typically, the substrate is one that is substantially planar, e.g., so that polymers, metals, and the like can be patterned on the substrate. In addition, the substrate typically contains other electronic components, for example, in electrical communication with one or more electrodes, or otherwise forming part of an electrical circuit. Examples include, but are not limited to, transistors such as field effect transistors, resistors, capacitors, inductors, diodes, integrated circuits, etc. In some cases, some of these may also comprise nanoscale wires.

In some embodiments, a portion of the substrate can be oxidized, e.g., forming SiO₂ and/or Si₃N₄ on a portion of the substrate, which may facilitate subsequent addition of materials (metals, polymers, etc.) to the substrate. In some cases, the oxidized portion may form a layer of material on the substrate, e.g., having a thickness of less than about 5 micrometers, less than about 4 micrometers, less than about 3 micrometers, less than about 2 micrometers, less than about 1 micrometer, less than about 900 nm, less than about 800 nm, less than about 700 nm, less than about 600 nm, less than about 500 nm, less than about 400 nm, less than about 300 nm, less than about 200 nm, less than about 100 nm, etc. In some cases, the substrate can include a sacrificial material that may then be removed, e.g., to cause the holding member to hold the nanoscale wire at an angle away from the substrate. As noted, in some cases, portions of the holding member and/or electrodes may be deposited such that, upon removal of the sacrificial material, stresses within the holding member may cause warping or bending such that the holding member holds the nanoscale wire at an angle away from the substrate.

In one set of embodiments, for example, at least a portion of the sacrificial material can be exposed to an etchant able to remove the sacrificial material. For example, if the sacrificial material is a metal such as nickel, a suitable etchant (for example, a metal etchant such as a nickel etchant, acetone, etc.) may be used to remove the sacrificial metal. Many such etchants may be readily obtained commercially.

As mentioned, in certain aspects, the probe containing the nanoscale wires may be inserted into the tissue of a subject. The tissues usually comprise more than one cell, and may define part of all of an organ in some cases. The probe may be used, as discussed, for applying an electrical signal to the tissue, and/or for determining a property of the tissue. In some embodiments, the nanoscale wires may be inserted into a cell using the probe, or positioned outside the cell, using the probe for example, to determine or stimulate intracellular or extracellular functions of the cell.

The property may be, for example, a chemical property, an electrical property, a mechanical property, or the like. Techniques for using nanoscale wires for determining such properties, e.g., using a reaction entity, are discussed in more detail below. In some cases, the tissue that the probe is inserted into may be an electrically-active tissue. The electrically-active tissue can be a tissue that produces or is sensitive to electrical signals for normal biological function. Non-limiting examples of electrically-active tissues include the heart, the brain, the nervous system (e.g., the central and/or peripheral nervous systems), muscles, sensory organs such as the eye or the ear, the enteric nervous system, etc. In some cases, specific cells within the nervous system (e.g., glial cells, astrocytes, neurons, etc.) may be determined and/or stimulated. However, in other embodiments, the tissue is not necessarily electrically active. For example, the probe may be used to determine a chemical property or a mechanical property, or an electrical signal may be applied to the tissue, e.g., to stimulate the tissue. In addition, as previously discussed, in some embodiments, the probe may be inserted into other materials.

In addition, in some embodiments, more than one probe may be used, for example, inserted into the same or different tissues. For instance, a first probe may be inserted into a first tissue and a second probe may be inserted into the first tissue, into a different location within the first tissue, into a second tissue, etc. As a non-limiting example, multiple nerves may be simultaneously determined and/or stimulated using various probes. For instance, a first probe may be inserted into a first region of the brain and a second probe may be inserted into a second region of the brain (e.g., without removing the first probe from the brain).

For example, in some embodiments, some or all of the nanoscale wires may be used to determine a property, such as a chemical property, a mechanical property, an electrical property, or the like. In some cases, the property may be determined at a relatively high resolution, e.g., due to the placement of nanoscale wires on the probe. For example, one or more nanoscale wires may be present within an electronic circuit as a component of a transistor. In addition, in certain embodiments, such determinations may be transmitted and/or recorded, e.g., for later use and/or analysis.

Thus, for example, a property such as a chemical property, a mechanical property, an electrical property, etc. can be determined at a resolution of less than about 2 mm, less than about 1 mm, less than about 500 micrometers, less than about 300 micrometers, less than about 100 micrometers, less than about 50 micrometers, less than about 30 micrometers, or less than about 10 micrometers, etc. In addition, in some cases, such properties can be determined and/or recorded as a function of time. Thus, for example, such properties can be determined at a time resolution of less than about 1 min, less than about 30 s, less than about 15 s, less than about 10 s, less than about 5 s, less than about 3 s, less than about 1 s, less than about 500 ms, less than about 300 ms, less than about 100 ms, less than about 50 ms, less than about 30 ms, less than about 10 ms, less than about 5 ms, less than about 3 ms, less than about 1 ms, etc.

In addition, in some embodiments, a tissue, and/or portions of a tissue (or another material), may be electrically stimulated using the nanoscale wires. For example, all or a subset of the nanoscale wires may be electrically stimulated, e.g., by using an external electrical system, such as a computer. Thus, for example, a single nanoscale wire, a group of nanoscale wires, or substantially all of the nanoscale wires can be electrically stimulated, depending on the particular application. In some cases, such nanoscale wires can be stimulated in a particular pattern, e.g., to cause cardiac or muscle cells to contract or beat in a particular pattern (for example, as part of a prosthetic or a pacemaker), to cause the firing of neurons with a particular pattern, to monitor the status of an implanted tissue within a subject, or the like.

In addition, in various embodiments, a nanoscale wire may stimulate and/or determine the properties of one, or more than one cell or tissue (or another material). For example, a nanoscale wire may be in physical contact with a single cell, or a group of cells. In addition, in some cases, due to their small size, more than one nanoscale wire may be positioned in physical contact with a single cell. Thus, for example, different sites from a single cell (e.g., a neuron) may be determined or stimulated simultaneously. In addition, in some embodiments, due to their small size, specific locations of a cell may be determined and/or stimulated, e.g., without stimulating the entire cell. For example, in a nerve cell, axons, dendrites, dendritic spines, etc. may be individually determined or stimulated.

As another example, in some cases, some or all of the nanoscale wires may be used to determine heart rate, e.g., by determining mechanical deflections of the nanoscale wires. In some embodiments, this can be determined as a change in conductance, which can be recorded. In some cases, for example, periodic changes in the conductance of a nanoscale wire may be determined, which may be due to the heart rate or blood flow. For instance, the diameter of a red blood cell is approximately about 8 micrometer, and red blood cells coming into contact with the nanoscale wires could cause mechanical deflections in the nanoscale wire, which could be determined as changes in conductance.

As mentioned, any nanoscale wire can be used. Non-limiting examples of suitable nanoscale wires include carbon nanotubes, nanorods, nanowires, organic and inorganic conductive and semiconducting polymers, metal nanoscale wires, semiconductor nanoscale wires (for example, formed from silicon), and the like. If carbon nanotubes are used, they may be single-walled and/or multi-walled, and may be metallic and/or semiconducting in nature. Other conductive or semiconducting elements that may not be nanoscale wires, but are of various small nanoscopic-scale dimension, also can be used in certain embodiments.

In general, a “nanoscale wire” (also known herein as a “nanoscopic-scale wire” or “nanoscopic wire”) generally is a wire or other nanoscale object, that at any point along its length, has at least one cross-sectional dimension and, in some embodiments, two orthogonal cross-sectional dimensions (e.g., a diameter) of less than 1 micrometer, less than about 500 nm, less than about 200 nm, less than about 150 nm, less than about 100 nm, less than about 70, less than about 50 nm, less than about 20 nm, less than about 10 nm, less than about 5 nm, than about 2 nm, or less than about 1 nm. In some embodiments, the nanoscale wire is generally cylindrical. In other embodiments, however, other shapes are possible; for example, the nanoscale wire can be faceted, i.e., the nanoscale wire may have a polygonal cross-section. The cross-section of a nanoscale wire can be of any arbitrary shape, including, but not limited to, circular, square, rectangular, annular, polygonal, or elliptical, and may be a regular or an irregular shape.

The nanoscale wire can also be solid or hollow.

In some cases, the nanoscale wire has one dimension that is substantially longer than the other dimensions of the nanoscale wire. For example, the nanoscale wire may have a longest dimension that is at least about 1 micrometer, at least about 3 micrometers, at least about 5 micrometers, or at least about 10 micrometers or about 20 micrometers in length, and/or the nanoscale wire may have an aspect ratio (longest dimension to shortest orthogonal dimension) of greater than about 2:1, greater than about 3:1, greater than about 4:1, greater than about 5:1, greater than about 10:1, greater than about 25:1, greater than about 50:1, greater than about 75:1, greater than about 100:1, greater than about 150:1, greater than about 250:1, greater than about 500:1, greater than about 750:1, or greater than about 1000:1 or more in some cases.

In some embodiments, a nanoscale wire are substantially uniform, or have a variation in average diameter of the nanoscale wire of less than about 30%, less than about 25%, less than about 20%, less than about 15%, less than about 10%, or less than about 5%. For example, the nanoscale wires may be grown from substantially uniform nanoclusters or particles, e.g., colloid particles. See, e.g., U.S. Pat. No. 7,301,199, issued Nov. 27, 2007, entitled “Nanoscale Wires and Related Devices,” by Lieber, et al., incorporated herein by reference in its entirety. In some cases, the nanoscale wire may be one of a population of nanoscale wires having an average variation in diameter, of the population of nanowires, of less than about 30%, less than about 25%, less than about 20%, less than about 15%, less than about 10%, or less than about 5%.

In some embodiments, a nanoscale wire has a conductivity of or of similar magnitude to any semiconductor or any metal. The nanoscale wire can be formed of suitable materials, e.g., semiconductors, metals, etc., as well as any suitable combinations thereof. In some cases, the nanoscale wire will have the ability to pass electrical charge, for example, being electrically conductive. For example, the nanoscale wire may have a relatively low resistivity, e.g., less than about 10⁻³ Ohm m, less than about 10⁻⁴ Ohm m, less than about 10⁻⁶ Ohm m, or less than about 10⁻⁷ Ohm m. The nanoscale wire can, in some embodiments, have a conductance of at least about 1 microsiemens, at least about 3 microsiemens, at least about 10 microsiemens, at least about 30 microsiemens, or at least about 100 microsiemens.

The nanoscale wire can be solid or hollow, in various embodiments. As used herein, a “nanotube” is a nanoscale wire that is hollow, or that has a hollowed-out core, including those nanotubes known to those of ordinary skill in the art. As another example, a nanotube may be created by creating a core/shell nanowire, then etching away at least a portion of the core to leave behind a hollow shell. Accordingly, in one set of embodiments, the nanoscale wire is a non-carbon nanotube. In contrast, a “nanowire” is a nanoscale wire that is typically solid (i.e., not hollow). Thus, in one set of embodiments, the nanoscale wire may be a semiconductor nanowire, such as a silicon nanowire.

In one set of embodiment, a nanoscale wire may comprise or consist essentially of a metal. Non-limiting examples of potentially suitable metals include aluminum, gold, silver, copper, molybdenum, tantalum, titanium, nickel, tungsten, chromium, or palladium. In another set of embodiments, a nanoscale wire comprises or consists essentially of a semiconductor. Typically, a semiconductor is an element having semiconductive or semi-metallic properties (i.e., between metallic and non-metallic properties). An example of a semiconductor is silicon. Other non-limiting examples include elemental semiconductors, such as gallium, germanium, diamond (carbon), tin, selenium, tellurium, boron, or phosphorous. In other embodiments, more than one element may be present in the nanoscale wire as the semiconductor, for example, gallium arsenide, gallium nitride, indium phosphide, cadmium selenide, etc. Still other examples include a Group II-VI material (which includes at least one member from Group II of the Periodic Table and at least one member from Group VI, for example, ZnS, ZnSe, ZnSSe, ZnCdS, CdS, or CdSe), or a Group III-V material (which includes at least one member from Group III and at least one member from Group V, for example GaAs, GaP, GaAsP, InAs, InP, AlGaAs, or InAsP).

In certain embodiments, the semiconductor can be undoped or doped (e.g., p-type or n-type). For example, in one set of embodiments, a nanoscale wire may be a p-type semiconductor nanoscale wire or an n-type semiconductor nanoscale wire, and can be used as a component of a transistor such as a field effect transistor (“FET”). For instance, the nanoscale wire may act as the “gate” of a source-gate-drain arrangement of a FET, while metal leads or other conductive pathways (as discussed herein) are used as the source and drain electrodes.

In some embodiments, a dopant or a semiconductor may include mixtures of Group IV elements, for example, a mixture of silicon and carbon, or a mixture of silicon and germanium. In other embodiments, the dopant or the semiconductor may include a mixture of a Group III and a Group V element, for example, BN, BP, BAs, AlN, AlP, AlAs, AlSb, GaN, GaP, GaAs, GaSb, InN, InP, InAs, or InSb. Mixtures of these may also be used, for example, a mixture of BN/BP/BAs, or BN/AlP. In other embodiments, the dopants may include alloys of Group III and Group V elements. For example, the alloys may include a mixture of AlGaN, GaPAs, InPAs, GaInN, AlGaInN, GaInAsP, or the like. In other embodiments, the dopants may also include a mixture of Group II and Group VI semiconductors. For example, the semiconductor may include ZnO, ZnS, ZnSe, ZnTe, CdS, CdSe, CdTe, HgS, HgSe, HgTe, BeS, BeSe, BeTe, MgS, MgSe, or the like. Alloys or mixtures of these dopants are also be possible, for example, (ZnCd)Se, or Zn(SSe), or the like. Additionally, alloys of different groups of semiconductors may also be possible, for example, a combination of a Group II-Group VI and a Group III-Group V semiconductor, for example, (GaAs)ₓ(ZnS)₁₋ₓ. Other examples of dopants may include combinations of Group IV and Group VI elements, such as GeS, GeSe, GeTe, SnS, SnSe, SnTe, PbO, PbS, PbSe, or PbTe. Other semiconductor mixtures may include a combination of a Group I and a Group VII, such as CuF, CuCl, CuBr, Cut AgF, AgCl, AgBr, AgI, or the like. Other dopant compounds may include different mixtures of these elements, such as BeSiN₂, CaCN₂, ZnGeP₂, CdSnAs₂, ZnSnSb₂, CuGeP₃, CuSi₂P₃, Si₃N₄, Ge₃N₄, Al₂O₃, (Al, Ga, In)₂(S, Se, Te)₃, Al₂CO, (Cu, Ag)(Al, Ga, In, Tl, Fe)(S, Se, Te)₂ and the like.

The doping of the semiconductor to produce a p-type or n-type semiconductor may be achieved via bulk-doping in certain embodiments, although in other embodiments, other doping techniques (such as ion implantation) can be used. Many such doping techniques that can be used will be familiar to those of ordinary skill in the art, including both bulk doping and surface doping techniques. A bulk-doped article (e.g. an article, or a section or region of an article) is an article for which a dopant is incorporated substantially throughout the crystalline lattice of the article, as opposed to an article in which a dopant is only incorporated in particular regions of the crystal lattice at the atomic scale, for example, only on the surface or exterior. For example, some articles are typically doped after the base material is grown, and thus the dopant only extends a finite distance from the surface or exterior into the interior of the crystalline lattice. It should be understood that “bulk-doped” does not define or reflect a concentration or amount of doping in a semiconductor, nor does it necessarily indicate that the doping is uniform. “Heavily doped” and “lightly doped” are terms the meanings of which are clearly understood by those of ordinary skill in the art. In some embodiments, one or more regions comprise a single monolayer of atoms (“delta-doping”). In certain cases, the region may be less than a single monolayer thick (for example, if some of the atoms within the monolayer are absent). As a specific example, the regions may be arranged in a layered structure within the nanoscale wire, and one or more of the regions can be delta-doped or partially delta-doped.

Accordingly, in one set of embodiments, the nanoscale wires may include a heterojunction, e.g., of two regions with dissimilar materials or elements, and/or the same materials or elements but at different ratios or concentrations. The regions of the nanoscale wire may be distinct from each other with minimal cross-contamination, or the composition of the nanoscale wire can vary gradually from one region to the next. The regions may be both longitudinally arranged relative to each other, or radially arranged (e.g., as in a core/shell arrangement) on the nanoscale wire. Each region may be of any size or shape within the wire. The junctions may be, for example, a p/n junction, a p/p junction, an n/n junction, a p/i junction (where i refers to an intrinsic semiconductor), an n/i junction, an i/i junction, or the like. The junction can also be a Schottky junction in some embodiments. The junction may also be, for example, a semiconductor/semiconductor junction, a semiconductor/metal junction, a semiconductor/insulator junction, a metal/metal junction, a metal/insulator junction, an insulator/insulator junction, or the like. The junction may also be a junction of two materials, a doped semiconductor to a doped or an undoped semiconductor, or a junction between regions having different dopant concentrations. The junction can also be a defected region to a perfect single crystal, an amorphous region to a crystal, a crystal to another crystal, an amorphous region to another amorphous region, a defected region to another defected region, an amorphous region to a defected region, or the like. More than two regions may be present, and these regions may have unique compositions or may comprise the same compositions. As one example, a wire can have a first region having a first composition, a second region having a second composition, and a third region having a third composition or the same composition as the first composition. Non-limiting examples of nanoscale wires comprising heterojunctions (including core/shell heterojunctions, longitudinal heterojunctions, etc., as well as combinations thereof) are discussed in U.S. Pat. No. 7,301,199, issued Nov. 27, 2007, entitled “Nanoscale Wires and Related Devices,” by Lieber, et al., incorporated herein by reference in its entirety.

In some embodiments, the nanoscale wire is a bent or a kinked nanoscale wire. A kink is typically a relatively sharp transition or turning between a first substantially straight portion of a wire and a second substantially straight portion of a wire. For example, a nanoscale wire may have 1, 2, 3, 4, or 5 or more kinks. In some cases, the nanoscale wire is formed from a single crystal and/or comprises or consists essentially of a single crystallographic orientation, for example, a <110> crystallographic orientation, a <112> crystallographic orientation, or a <1120> crystallographic orientation. It should be noted that the kinked region need not have the same crystallographic orientation as the rest of the semiconductor nanoscale wire. In some embodiments, a kink in the semiconductor nanoscale wire may be at an angle of about 120o or a multiple thereof. The kinks can be intentionally positioned along the nanoscale wire in some cases. For example, a nanoscale wire may be grown from a catalyst particle by exposing the catalyst particle to various gaseous reactants to cause the formation of one or more kinks within the nanoscale wire. Non-limiting examples of kinked nanoscale wires, and suitable techniques for making such wires, are disclosed in International Patent Application No. PCT/US2010/050199, filed Sep. 24, 2010, entitled “Bent Nanowires and Related Probing of Species,” by Tian, et al., published as WO 2011/038228 on Mar. 31, 2011, incorporated herein by reference in its entirety.

In one set of embodiments, the nanoscale wire is formed from a single crystal, for example, a single crystal nanoscale wire comprising a semiconductor. A single crystal item may be formed via covalent bonding, ionic bonding, or the like, and/or combinations thereof. While such a single crystal item may include defects in the crystal in some cases, the single crystal item is distinguished from an item that includes one or more crystals, not ionically or covalently bonded, but merely in close proximity to one another.

In some embodiments, the nanoscale wires used herein are individual or free-standing nanoscale wires. For example, an “individual” or a “free-standing” nanoscale wire may, at some point in its life, not be attached to another article, for example, with another nanoscale wire, or the free-standing nanoscale wire may be in solution. This is in contrast to nanoscale features etched onto the surface of a substrate, e.g., a silicon wafer, in which the nanoscale features are never removed from the surface of the substrate as a free-standing article. This is also in contrast to conductive portions of articles which differ from surrounding material only by having been altered chemically or physically, in situ, i.e., where a portion of a uniform article is made different from its surroundings by selective doping, etching, etc. An “individual” or a “free-standing” nanoscale wire is one that can be (but need not be) removed from the location where it is made, as an individual article, and transported to a different location and combined with different components to make a functional device such as those described herein and those that would be contemplated by those of ordinary skill in the art upon reading this disclosure.

The nanoscale wire, in some embodiments, may be responsive to a property external of the nanoscale wire, e.g., a chemical property, an electrical property, a physical property, etc. Such determination may be qualitative and/or quantitative, and such determinations may also be recorded, e.g., for later use. For example, in one set of embodiments, the nanoscale wire may be responsive to voltage. For instance, the nanoscale wire may exhibits a voltage sensitivity of at least about 5 microsiemens/V; by determining the conductivity of a nanoscale wire, the voltage surrounding the nanoscale wire may thus be determined. In other embodiments, the voltage sensitivity can be at least about 10 microsiemens/V, at least about 30 microsiemens/V, at least about 50 microsiemens/V, or at least about 100 microsiemens/V. Other examples of electrical properties that can be determined include resistance, resistivity, conductance, conductivity, impendence, or the like.

As another example, a nanoscale wire may be responsive to a chemical property of the environment surrounding the nanoscale wire. For example, an electrical property of the nanoscale wire can be affected by a chemical environment surrounding the nanoscale wire, and the electrical property can be thereby determined to determine the chemical environment surrounding the nanoscale wire. As a specific non-limiting example, the nanoscale wires may be sensitive to pH or hydrogen ions. Further non-limiting examples of such nanoscale wires are discussed in U.S. Pat. No. 7,129,554, filed Oct. 31, 2006, entitled “Nanosensors,” by Lieber, et al., incorporated herein by reference in its entirety.

As a non-limiting example, the nanoscale wire may have the ability to bind to an analyte indicative of a chemical property of the environment surrounding the nanoscale wire (e.g., hydrogen ions for pH, or concentration for an analyte of interest), and/or the nanoscale wire may be partially or fully functionalized, i.e. comprising surface functional moieties, to which an analyte is able to bind, thereby causing a determinable property change to the nanoscale wire, e.g., a change to the resistivity or impedance of the nanoscale wire. The binding of the analyte can be specific or non-specific. Functional moieties may include simple groups, selected from the groups including, but not limited to, —OH, —CHO, —COOH, —SO₃H, —CN, —NH₂, —SH, —COSH, —COOR, halide; biomolecular entities including, but not limited to, amino acids, proteins, sugars, DNA, antibodies, antigens, and enzymes; grafted polymer chains with chain length less than the diameter of the nanowire core, selected from a group of polymers including, but not limited to, polyamide, polyester, polyimide, polyacrylic; a shell of material comprising, for example, metals, semiconductors, and insulators, which may be a metallic element, an oxide, an sulfide, a nitride, a selenide, a polymer and a polymer gel. A non-limiting example of a protein is PSA (prostate specific antigen), which can be determined, for example, by modifying the nanoscale wires by binding monoclonal antibodies for PSA (Abl) thereto. See, e.g., U.S. Pat. No. 8,232,584, issued Jul. 31, 2012, entitled “Nanoscale Sensors,” by Lieber, et al., incorporated herein by reference in its entirety.

In some embodiments, a reaction entity may be bound to a surface of the nanoscale wire, and/or positioned in relation to the nanoscale wire such that the analyte can be determined by determining a change in a property of the nanoscale wire. The “determination” may be quantitative and/or qualitative, depending on the application, and in some cases, the determination may also be analyzed, recorded for later use, transmitted, or the like. The term “reaction entity” refers to any entity that can interact with an analyte in such a manner to cause a detectable change in a property (such as an electrical property) of a nanoscale wire. The reaction entity may enhance the interaction between the nanowire and the analyte, or generate a new chemical species that has a higher affinity to the nanowire, or to enrich the analyte around the nanowire. The reaction entity can comprise a binding partner to which the analyte binds. The reaction entity, when a binding partner, can comprise a specific binding partner of the analyte. For example, the reaction entity may be a nucleic acid, an antibody, a sugar, a carbohydrate or a protein. Alternatively, the reaction entity may be a polymer, catalyst, or a quantum dot. A reaction entity that is a catalyst can catalyze a reaction involving the analyte, resulting in a product that causes a detectable change in the nanowire, e.g. via binding to an auxiliary binding partner of the product electrically coupled to the nanowire. Another exemplary reaction entity is a reactant that reacts with the analyte, producing a product that can cause a detectable change in the nanowire. The reaction entity can comprise a shell on the nanowire, e.g. a shell of a polymer that recognizes molecules in, e.g., a gaseous sample, causing a change in conductivity of the polymer which, in turn, causes a detectable change in the nanowire.

The term “binding partner” refers to a molecule that can undergo binding with a particular analyte, or “binding partner” thereof, and includes specific, semi-specific, and non-specific binding partners as known to those of ordinary skill in the art. The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen. Other examples include, nucleic acids that specifically bind (hybridize) to their complement, antibodies specifically bind to their antigen, and the like. The binding may be by one or more of a variety of mechanisms including, but not limited to ionic interactions, and/or covalent interactions, and/or hydrophobic interactions, and/or van der Waals interactions, etc.

The antibody may be any protein or glycoprotein comprising or consisting essentially of one or more polypeptides substantially encoded by immunoglobulin genes or fragments of immunoglobulin genes. Examples of recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′₂, a dimer of Fab which itself is a light chain joined to VHCH1 by a disulfide bond. The F(ab)′₂ may be reduced under mild conditions to break the disulfide linkage in the hinge region thereby converting the (Fab)₂ dimer into an Fab′ monomer. The Fab′ monomer is essentially a Fab with part of the hinge region. While various antibody fragments are defined in terms of the digestion of an intact antibody, one of skill will appreciate that such fragments may be synthesized de novo either chemically, by utilizing recombinant DNA methodology, or by “phage display” methods. Non-limiting examples of antibodies include single chain antibodies, e.g., single chain Fv (scFv) antibodies in which a variable heavy and a variable light chain are joined together (directly or through a peptide linker) to form a continuous polypeptide.

The following documents are incorporated herein by reference in their entireties: U.S. Pat. No. 7,211,464, issued May 1, 2007, entitled “Doped Elongated Semiconductors, Growing Such Semiconductors, Devices Including Such Semiconductors, and Fabricating Such Devices,” by Lieber, et al.; and U.S. Pat. No. 7,301,199, issued Nov. 27, 2007, Ser. No. 12/308,207, filed Ser. No. 10/588,833, filed Aug. 9, 2006, entitled “Nanostructures Containing Metal-Semiconductor Compounds,” by Lieber, et al., published as U.S. Patent Application Publication No. 2009/0004852 on Jan. 1, 2009; U.S. patent application Ser. No. 10/995,075, filed Nov. 22, 2004, entitled “Nanoscale Arrays, Robust Nanostructures, and Related Devices,” by Whang, et al., published as 2005/0253137 on Nov. 17, 2005; U.S. patent application Ser. No. 11/629,722, filed Dec. 15, 2006, entitled “Nanosensors,” by Wang, et al., published as U.S. Patent Application Publication No. 2007/0264623 on Nov. 15, 2007; International Patent Application No. PCT/US2007/008540, filed Apr. 6, 2007, entitled “Nanoscale Wire Methods and Devices,” by Lieber et al., published as WO 2007/145701 on Dec. 21, 2007; U.S. patent application Ser. No. ______ Dec. 9, 2008, entitled “Nanosensors and Related Technologies,” by Lieber, et al.; U.S. Pat. No. 8,232,584, issued Jul. 31, 2012, entitled “Nanoscale Sensors,” by Lieber, et al.; U.S. patent application Ser. No. 12/312,740, filed May 22, 2009, entitled “High-Sensitivity Nanoscale Wire Sensors,” by Lieber, et al., published as U.S. Patent Application Publication No. 2010/0152057 on Jun. 17, 2010; and International Patent Application No. PCT/US2010/050199, filed Sep. 24, 2010, entitled “Bent Nanowires and Related Probing of Species,” by Tian, et al., published as WO 2011/038228 on Mar. 31, 2011.

The following examples are intended to illustrate certain embodiments of the present invention, but do not exemplify the full scope of the invention.

### Example 1

The probes described in this example includes a substrate and a pattern of metal leads towards its tip. The substrate comprises an array of single or multiple semiconductive nanowire or nanotube field effect transistors. Each semiconductive FET is connected to a source and a drain metal electrodes, which are connected to the metal leads. The FETs are configured either on the substrate plane or out of it (in a 3D manner) in order to get better interface with cells. The probe records the change of potential as change of conductance, as the extra or intracellular solution serve as gate.

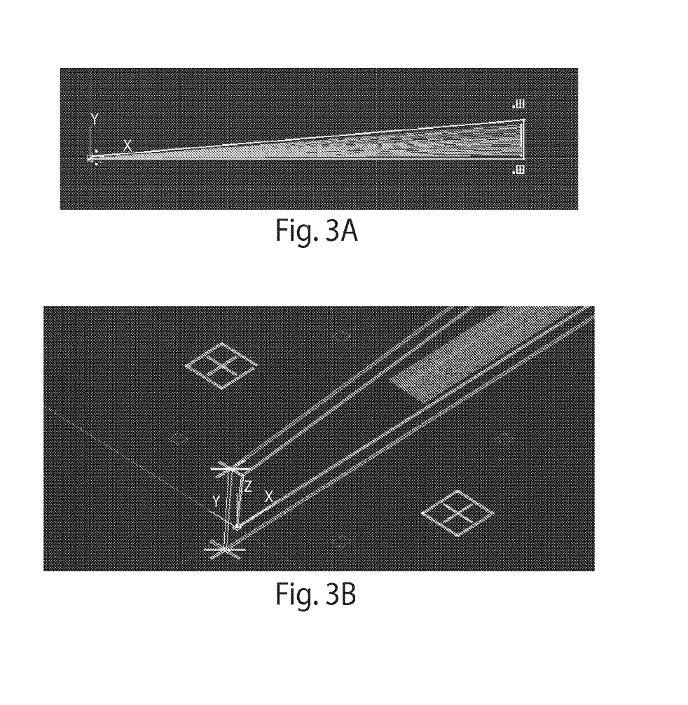

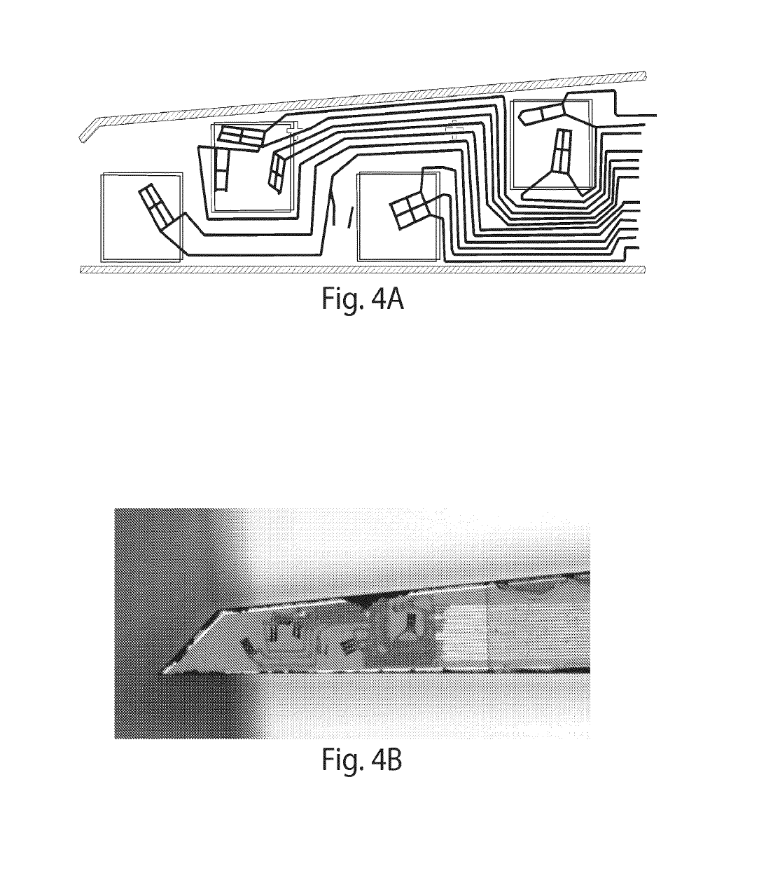

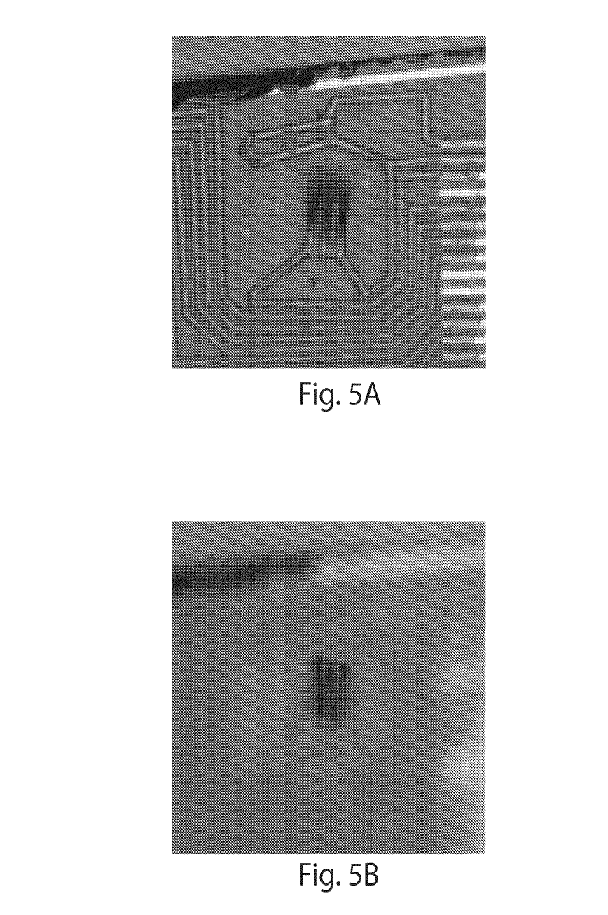

Examples of such a probe are shown in FIGS. 3-5. FIG. 3 illustrates various views of one example of a probe. The portion of the probe that is inserted into the subject has a length of 900 micrometers and a width of 150 micrometers. However, as discussed herein, other dimensions of the probe are also possible. FIG. 4 illustrates the design and fabrication of several silicon nanowires on the probe. FIG. 5 illustrates a close-up of one of the nanowires, illustrating that it is held at an angle away from the surface of the probe, e.g., by a suitable holding member.

### Example 2

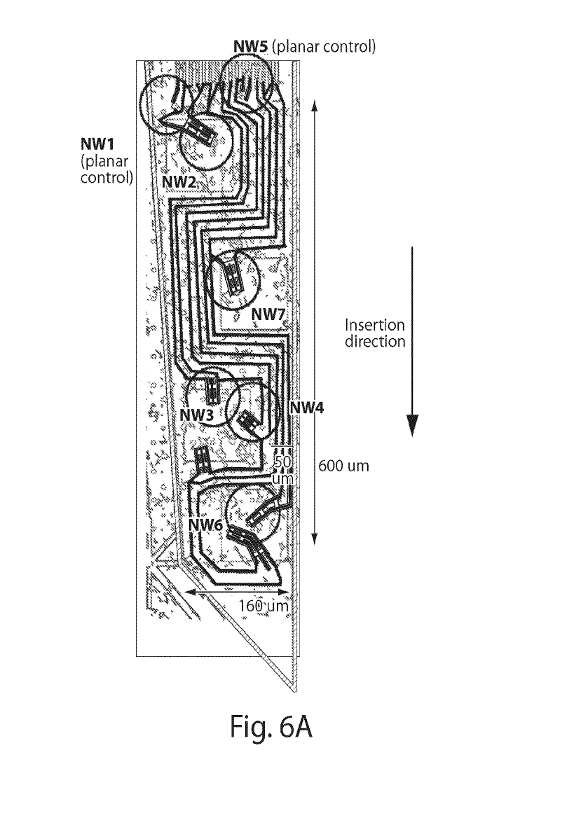

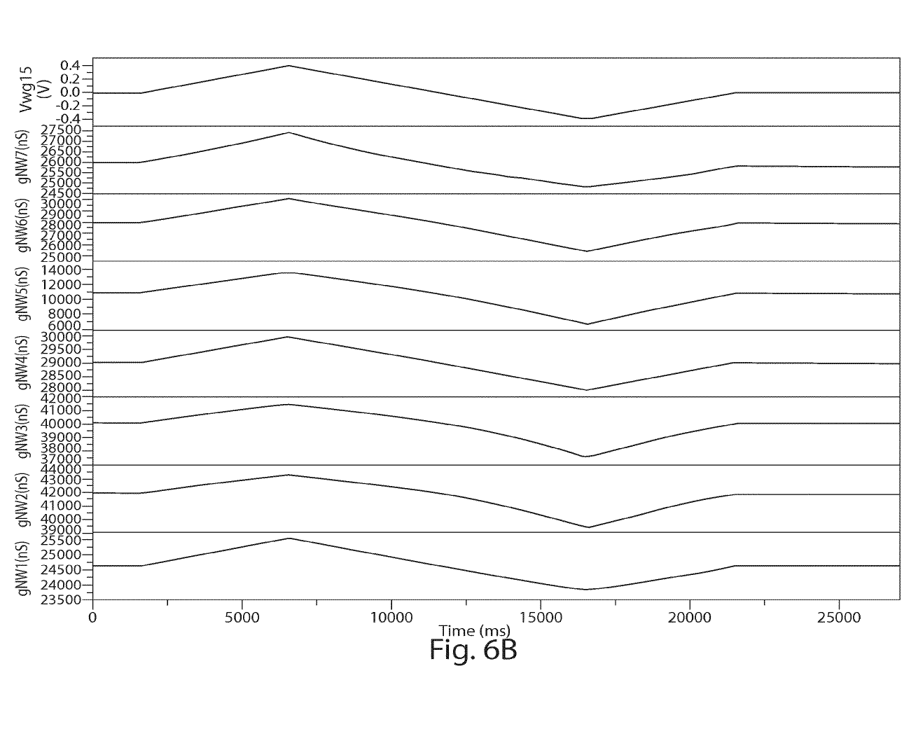

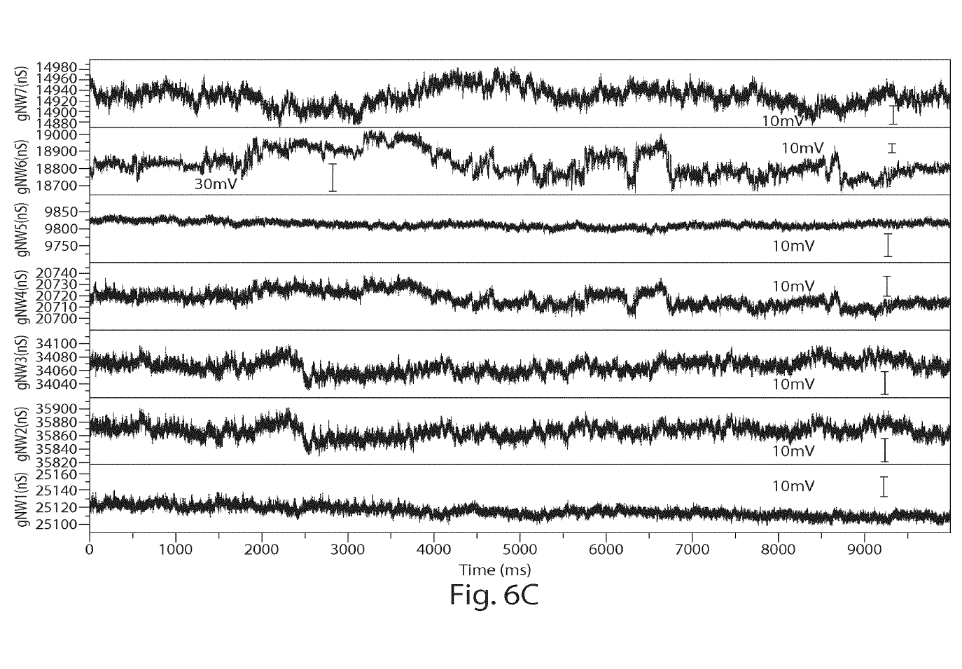

This example illustrates in vivo recordings using a probe as was discussed in the previous example. FIG. 6A illustrates the portion of the probe insert into the subject, with the nanowires circled. Two of the nanowires, however, were not angled away from the surface of the probe (identified as planar control). FIGS. 6B-6C illustrates a water gate experiment that was used to determine the sensitivity of the devices inside the somatosensory cortex of a live rat (in-vivo). For instance, FIG. 6C illustrates simultaneous recordings from the cortex of a rat in vivo using 7 nanowires. The conductance changes which were measured were converted to voltage according to device sensitivities determined in the water gate experiments in FIG. 6B. Different behaviors were observed for each of the nanowires, which appeared to be relatively uncorrelated, thereby illustrating that each nanowire experienced a different electrical environment. Thus, for example, some nanowires appeared to be in electrically active regions of the cortex, and some of the nanowires appeared to be monitoring relatively quick action potentials or other electrical events.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article for insertion into a tissue, comprising:
a substrate constructed and arranged for insertion into tissue, a plurality of nanoscale wires, and a plurality of electrical connectors in electrical communication with the plurality of nanoscale wires.

2. The article of claim 1, wherein at least some of the nanoscale wires comprise a semiconductor.

3. The article of claim 1, wherein at least some of the nanoscale wires comprise silicon.

4-9. (canceled)

10. The article of claim 1, wherein the substrate has a width of no more than about 200 micrometers.

11. The article of claim 1, wherein the substrate has a thickness of no more than about 10 micrometers.

12. The article of claim 1, wherein the substrate has an angled tip.

13. The article of claim 12, wherein the angled tip defines an angle in the plane of no more than about 90°.

14. (canceled)

15. The article of claim 1, wherein at least a portion of the substrate is biodegradable.

16. The article of claim 1, wherein at least a portion of the substrate comprises silk.

17. (canceled)

18. The article of claim 1, wherein substantially each of the nanoscale wires is individually addressable via the plurality of electrical connectors.

19-20. (canceled)

21. The article of claim 1, wherein at least some of the nanoscale wires form part of a field effect transistor.

22. The article of claim 1, wherein at least some of the nanoscale wires is responsive to an electrical property external to the nanoscale wire.

23. The article of claim 1, wherein at least one of the nanoscale wires exhibits a voltage sensitivity of at least about 5 microsiemens/V.

24. The article of claim 1, wherein at least one of the nanoscale wires is in electrical communication with a reaction entity such that an interaction between the reaction entity and an analyte causes a detectable change in a property of the nanoscale wire.

25-32. (canceled)

33. A method, comprising:
inserting a substrate comprising a plurality of nanoscale wires into a tissue of a subject.

34-35. (canceled)

36. The method of claim 33, wherein the substrate comprises a tip is constructed and arranged for insertion into tissue.

37. The method of claim 33, comprising inserting the substrate into the brain of a subject.

38. The method of claim 33, comprising inserting the substrate into the heart of a subject.

39. The method of claim 33, further comprising determining an electrical property of at least some of the nanoscale wires.

40-44. (canceled)

45. The method of claim 33, further comprising removing at least a portion of the substrate without removing the plurality of nanoscale wires from the electrically-active tissue

46. The method of claim 45, wherein at least a portion of the substrate is removed by chemical degradation.

47-48. (canceled)

49. A method, comprising:
externally delivering an electrical stimulus to a tissue within a subject via a nanoscale wire inserted therein.

50-71. (canceled)
